(19) United States
(12) Patent Application Publication
(10) Pub. No.: US 2023/0210986 A1
(43) Pub. Date: Jul. 6, 2023

(54) NON-PSYCHOACTIVE CANNABINOIDS AS ADJUVANTS TO ENHANCE MUCOSAL IMMUNITY

(71) Applicant: Immugen Pharma LLC, South Miami, FL (US)

(72) Inventor: Craig Travis, South Miami, FL (US)

(21) Appl. No.: 18/113,776

(22) Filed: Feb. 24, 2023

Related U.S. Application Data
(63) Continuation of application No. 17/216,819, filed on Mar. 30, 2021.

(51) Int. Cl. A61K 39/39

(52) U.S. Cl. CPC A61K 39/39; A61K 2039/55511

(57) ABSTRACT

Disclosed are methods and compositions related to the use cannabinoids as adjuvants for the accelerated induction and production of an antibody based immune response.

This application claims the benefit of U.S. Application No. 62/001,667, filed on Mar. 30, 2020, which is incorporated herein by reference in its entirety.

### I. BACKGROUND

The portals of entry for most pathogens that cause disease in humans and livestock are through the mucosal linings of the respiratory tract, gastrointestinal tract and in the case of sexually transmitted pathogens in humans, either the reproductive organs or rectal mucosa. Within the mucosa is the presence of the immune system that is there to capture, process and mount a response to limit and reduce the consequences of the infection. The response is immediate in the form of a series of events initiated by mucosally expressed host defense peptides that are based on conserved pathogen-associated molecular patterns that set off a cascade of events that result in an orchestrated immune response. The cellular sentinels of this innate response are the dendritic cells that capture and present the pathogen to lymphocytes resulting in the eventual destruction of the pathogen by an inflammatory cascade, the production of antibodies, memory for the antigen and eventually resolution. Sometimes, the response is dysregulated and results in severe morbidity and mortality.

The development of vaccines has had a profound effect in altering the spread of these diseases by exposing the immune system to components of a pathogen so that in the event of exposure to the disease-causing organism, the body will be prepared to disable and ultimately destroy it. However, there is a requirement that elements of the microorganism be identified, sequenced or purified in order to give it as a vaccine which takes time. Once a vaccine has been developed, it needs to be given in such a way as to produce its effects which usually requires an adjuvant which is defined as any substance that acts to accelerate, prolong, or enhance antigen-specific immune responses when used in combination with specific vaccine antigens.

All infections eventually result in the production of antibodies after the exposure that help to control the current infection and protect against future infections with the same microorganism if the host doesn't first succumb to a dysregulated immune response that is the result of an innate cellular response with its production of inflammatory cytokines. It is therefore necessary to accelerate the adaptive immune response with the induction of the IgA class of antibodies at the mucosal surfaces. Not only will the production of IgA antibodies bind to the specific epitopes that have been identified and processed by the host's immune response that will lead to its neutralization and elimination of the pathogen, but also act in a non-inflammatory manner since neither the secreted, monomeric form (sIgA) found in serum nor the secretory, polymeric form (S-IgA) found in mucosal secretions activate any of the three complement pathways that are inflammatory in nature. The expression of the IgA isotype also blocks the binding of the IgG and IgM isotypes to the antigen and thus prevents the complement-mediated inflammatory effects associated with these isotypes. What are needed are new adjuvants and treatments that can address these issues.

### II. SUMMARY

Disclosed are methods and compositions related to the use cannabinoids as adjuvants for the accelerated induction and production of an immune response.

In one aspect, disclosed are anti-microbial vaccine or therapeutic comprising a microbial antigen and a cannabinoid receptor agonist (such as, for example, a cannabinoid receptor 2 agonist including, but not limited to L759,633; L759,656; JWH-056, JWH-133;JWH-229; JWH-352; JWH-359; THC; and/or CBD. In one aspect, the cannabinoid receptor 2 agonist can comprise a Dibenzopyran.

Also disclosed herein are anti-microbial vaccines or therapeutics of any preceding aspect, wherein the microbial antigen comprises a bacterial antigen from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Actinobacillus pleuropneumoniae, Listeria monocytogenes, Listeria ivanovii, Brucella abortus, other Brucella species, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Helicobactor pylori, Pseudomonas aeruginosa, other Pseudomonas species, Haemophilus influenzae, Haemophilus ducreyi, other Hemophilus species, Clostridium tetani, Clostridium difficile, Clostridium perfringens,other Clostridium species, Yersinia pestis, Yersinia enterolitica, and other Yersinia species.

In one aspect, disclosed herein are anti-microbial vaccines or therapeutics of any preceding aspect, wherein the microbial antigen comprises a viral antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A, Influenza virus B, Measles virus, Polyomavirus, Human Papilomavirus, Respiratory syncytial virus, Adenovirus, Coxsackie virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed herein are anti-microbial vaccines or therapeutics of any preceding aspect, wherein the microbial antigen comprises a fungal antigen from a fungi selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

In one aspect disclosed herein are anti-microbial vaccines or therapeutics of any preceding aspect, wherein the microbial antigen comprises an antigen from a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

In one aspect disclosed herein are methods of enhancing an immune response in a subject to an antigen (including, but not limited mucosal immune responses to ongoing infections) comprising administering to the subject a cannabinoid receptor agonist or any of the anti-microbial vaccines or therapeutics of any preceding aspect. For example, disclosed herein are methods of enhancing an immune response in a subject to an antigen comprising administering to the subject a cannabinoid receptor agonist (such as, for example L759,633; L759,656; JWH-056; JWH-133;JWH-229; JWH-352; JWH-359; THC; and/or CBD) to the subject. For example, the immune responses that is enhanced comprises increased and/or more rapid production of Immunoglobulin (Ig) A (IgA). In some aspects, the enhanced immune response further comprises an increase in production of B cell activating factor (BAFF/BLyS), a proliferation-inducing ligand (APRIL), Thymic stromal lymphopoietin (TSLP), IL-33, IL-10, IL-4, IL-6, and TGF-β.

Also disclosed herein are methods of increasing the efficacy of a vaccine in a subject comprising administering to the subject any of the anti-microbial vaccines or therapeutics of any preceding aspect. For example, disclosed herein are methods of increasing the efficacy of a vaccine in a subject comprising administering to the subject a cannabinoid receptor agonist (such as, for example L759,633; L759,656; JWH-056; JWH-133;JWH-229; JWH-352; JWH-359; THC; and/or CBD) to the subject.

In one aspect, disclosed herein are methods of inducing in a subject a mucosal immune response to an antigen said method comprising administering to the subject any of the anti-microbial vaccines or therapeutics of any preceding aspect. For example, disclosed herein are methods of inducing in a subject a mucosal immune response to an antigen said method comprising administering to the subject a cannabinoid receptor agonist (such as, for example L759,633; L759,656; JWH-056; JWH-133;JWH-229; JWH-352; JWH-359; THC; and/or CBD) and the antigen.

In one aspect, disclosed herein are methods of treating, inhibiting, reducing, decreasing, ameliorating, attenuating, and/or preventing a microbial infection in a subject comprising administering to the subject any of the anti-microbial vaccines or therapeutics of any preceding aspect. For example, disclosed herein are methods of treating, inhibiting, reducing, decreasing, ameliorating, attenuating, and/or preventing a microbial infection in a subject comprising administering to the subject a cannabinoid receptor agonist (such as, for example L759,633; L759,656; JWH-056; JWH-133;JWH-229; JWH-352; JWH-359; THC; and/or CBD) and an antigen; wherein the antigen comprises a microbial antigen.

Also disclosed herein are methods of enhancing an immune response of any preceding aspect; method of increasing the efficacy of a vaccine of any preceding aspect; methods of increasing the immunogenicity of an antigen of any preceding aspect; methods of inducing in a subject a mucosal immune response to an antigen of any preceding aspect; and/or methods of treating, inhibiting, reducing, decreasing, ameliorating, attenuating, and/or preventing a microbial infection in a subject of any preceding aspect, wherein the cannabinoid receptor agonist and the antigen are administered in the same formulation, concurrently in separate formulations, wherein the cannabinoid receptor agonist is administered before administration of the antigen; and/or wherein the cannabinoid receptor agonist is administered after administration of the antigen.

Also disclosed herein are methods of enhancing an immune response of any preceding aspect; method of increasing the efficacy of a vaccine of any preceding aspect; methods of increasing the immunogenicity of an antigen of any preceding aspect; methods of inducing in a subject a mucosal immune response to an antigen of any preceding aspect; and/or methods of treating, inhibiting, reducing, decreasing, ameliorating, attenuating, and/or preventing a microbial infection in a subject of any preceding aspect, wherein the antigen comprises a bacterial antigen from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Actinobacillus pleuropneumoniae, Listeria monocytogenes, Listeria ivanovii, Brucella abortus, other Brucella species, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Helicobactor pylori, Pseudomonas aeruginosa, other Pseudomonas species, Haemophilus influenzae (including, but not limited to Haemophilus influenzae type b), Haemophilus ducreyi, other Hemophilus species, Clostridium tetani, Clostridium difficile, Clostridium perfringens,other Clostridium species, Yersinia pestis, Yersinia enterolitica, and other Yersinia species.

Also disclosed herein are methods of enhancing an immune response of any preceding aspect; method of increasing the efficacy of a vaccine of any preceding aspect; methods of increasing the immunogenicity of an antigen of any preceding aspect; methods of inducing in a subject a mucosal immune response to an antigen of any preceding aspect; and/or methods of treating, inhibiting, reducing, decreasing, ameliorating, attenuating, and/or preventing a microbial infection in a subject of any preceding aspect, wherein the antigen comprises a viral antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A, Influenza virus B, Measles virus, Polyomavirus, Human Papilomavirus, Respiratory syncytial virus, Adenovirus, Coxsackie virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In one aspect, disclosed herein are methods of enhancing an immune response of any preceding aspect; method of increasing the efficacy of a vaccine of any preceding aspect; methods of increasing the immunogenicity of an antigen of any preceding aspect; methods of inducing in a subject a mucosal immune response to an antigen of any preceding aspect; and/or methods of treating, inhibiting, reducing, decreasing, ameliorating, attenuating, and/or preventing a microbial infection in a subject of any preceding aspect, wherein the antigen comprises a fungal antigen from a fungi selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

Also disclosed herein are methods of enhancing an immune response of any preceding aspect; method of increasing the efficacy of a vaccine of any preceding aspect; methods of increasing the immunogenicity of an antigen of any preceding aspect; methods of inducing in a subject a mucosal immune response to an antigen of any preceding aspect; and/or methods of treating, inhibiting, reducing, decreasing, ameliorating, attenuating, and/or preventing a microbial infection in a subject of any preceding aspect, wherein the antigen comprises an antigen from a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

In one aspect, disclosed herein are methods of enhancing an immune response of any preceding aspect; method of increasing the efficacy of a vaccine of any preceding aspect; methods of increasing the immunogenicity of an antigen of any preceding aspect; methods of inducing in a subject a mucosal immune response to an antigen of any preceding aspect; and/or methods of treating, inhibiting, reducing, decreasing, ameliorating, attenuating, and/or preventing a microbial infection in a subject of any preceding aspect, wherein the antigen comprises a vaccine or an antigen from a portion of a vaccine selected from the group of vaccines to microbes including, but not limited to Vibrio cholerae (including, but not limited to DUKORAL1®, SHANCHOL®, EUVICHOL®, VAXCHORA®), Influenza type A and B virus (including, but not limited to FluMist™), Poliovirus (including, but not limited to Biopolio™ B1/3, and other oral polio vaccines—OPVs), Rotavirus (including, but not limited to ROTARIX® and ROTATEQ®), Salmonella typhimurium (including, but not limited to TYPHI VIVOTIF®), Adenovirus, Rabies virus (including, but note limited to RABORAL-V-RG), Bovine parainfluenza 3 (including, but not limited to Rispoval), bovine respiratory syncytial virus, Bordetella bronchiseptica (including, but not limited to NOBIVAC®), Canine parainfluenza virus, Newcastle disease virus (including, but not limited to Avinew NeO™) Diphtheria, Hepatitis B, Haemophilus influenzae type b, Human papillomavirus, Seasonal influenza, Measles, Mumps, Pertussis, Rubella, Pneumococcal disease, Poliomyelitis (Polio), Rotavirus, Tetanus, Tuberculosis (TB), Varicella, Cholera, Hepatitis A, Hepatitis E, Japanese encephalitis, Meningococcal disease, Polio (adult booster dose), Rabies, Tick-borne encephalitis, Typhoid fever, and/or Yellow fever.

### III. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

As used herein, “adjuvant” refers to any substance that acts to accelerate, prolong, or enhance antigen specific immune responses when used in combination with a the antigen against with antigen specific immune responses are sought.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts.

An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Using the Compositions

The endocannabinoid system with its production of specialized fatty acids in response to immune activation indicates that it plays an integral role in both innate as well as adaptive immunity through activation of both CB1 and CB2 receptors (G-protein receptors) which are expressed by all cell types in the immune system Since the rank order of CB2 receptors is B-cells >natural killer cells >>monocytes >PMNs >CD8 T cells >CD4 T cells it suggests a significant role in the earliest interaction between antigen presenting cells and lymphocytes. Although the CB1 receptor which is found predominantly in the central nervous system it is also found in immune cells but at a level which is 10-100 fold lower than the CB2 receptor. Ligand induced activation of the CB2 receptors results in enhanced migration of B cells, dendritic cells, NK cells, macrophages to the sites of infection/inflammation while inhibiting the migration of T cells. Activation of the CB2 receptors with tetrahydrocannabinol (THC) in T cells stimulated by allogeneic dendritic cells or with anti-CD3 and anti-CD28 antibodies resulted in a significant shift in the balance of T_{H}1 towards a T_{H}2 profile of helper T cells noted by a decrease of INF-γ by 51±18% (p≤0.05) and an increase of IL-4 to 105±11% of diluent control levels respectively. Moreover, the effects THC closely modeled the suppressive effects observed with IL-4 on re-stimulated T cells to depress IFN-γ in a CB2 dependent manner. Evidence from another study confirmed that addition of THC in a mixed lymphocyte reaction and highly purified T cells led to a downregulation of the Th1 cytokines Il-2, IFN-γ and Il-12 in a CB1 mediated fashion and the upregulation of IL-4 and Il-5 in a CB2 mediated fashion indicating an overall shift from a T_{H}1 to a T_{H}2 profile. It was later determined that in mice treated with THC following a Legionella challenge showed the upregulation of the T_{H}2 biasing factor GATA3 which is necessary for the production of IL-4 was mediated through a CB2 mechanism while the downregulation of IL12Rβ2, a marker of T_{H}1 polarization, was mediated by the CB1 receptor. Yet another group examined the effects of a CB2 specific ligand, JWH-133, in macrophages which were activated by LPS or Theiler's virus (TMEV) and discovered two mechanisms by which IL-12p40 was downregulated—a greater and sustained activation of ERK1/2 MAP kinase which lead to the differential production of IL-12 and IL-10 as well as the autocrine regulation of IL-12 by IL-10. With the use of selective inverse agonists they also established that the T helper biasing events mediated by macrophages is due, in part, to the activation of the CB2 receptors. Therefore, it appears that activation of the CB2 receptors may be the initiating process for the switch to T_{H}2 profile with the production of IL-4 in T-cells and IL-10 in both T cells and macrophages.

Another CB2 mediated event which provides evidence of the immunomodulatory properties of the endocannabinoid system is the upregulation of TFG-β by activated lymphocytes, yet this results in the downregulation of CB2 receptors in an autocrine mediated manner. A study involving both occasional and regular use of smoked marijuana showed that IL-2 was downregulated while IL-10, a T_{H}2 cytokine and TGF-β were upregulated. This increase in T_{H}2 and T_{H}3 cell activity which produces IL-4, IL-10 and TGF-β in turn would have a net anti-inflammatory and regulatory effect on the immune response resulting in the generation of T regulatory cells and possibly influence class switching from one antibody isotype to another, particularly IgA. Since the endocannabinoid system is essentially anti-inflammatory in nature, an enhancement of an IgA response is in keeping with this paradigm since SIgA itself is a non-inflammatory.

Human B cells isolated from PBMCs have the highest level of CB2 mRNA compared to other immune cells. Although higher concentrations (>1 M) of cannabinoid ligands have been shown to cause immunosuppression, the concentrations used are not physiologically relevant since affinity binding experiments show that low nanomolar concentrations of various ligands achieve high affinity binding for the receptor. Two different experimental conditions, one by cross-linking of anti-Igs activator to the surface of B cells and the other by the ligation of the CD40 antigen in the presence of the 10⁻⁸ test compound induced a 40% to 70% increase in thymidine uptake in B cells respectively. In the absence of costimulating agents, there was no stimulation of B-cell growth by the cannabinoids. It was also noted that there was a marked effect of the culture medium containing different concentrations of FCS: the optimal concentration which favored increased thymidine uptake was 0.25% to 0.5%, whereas 5% FCS almost totally abolished the effect of the ligands presumably due to the non-specific interactions with serum proteins and endocannabinoids present in the FCS. The addition of pertussis toxin confirmed that the action of the cannabinoids was mediated through the Gᵢ proteins of the receptor. Since the selective antagonistic effect of SR141716A to the CB1 receptor did not block the mRNA expression of the CB2 receptor nor the B-cell enhancing effects of the drug this group concluded that B-cell growth was a CB2 receptor mediated process. This was later confirmed when a selective CB2 receptor antagonist SR 144528A inhibited the proliferative response of virgin and GC B cells by a potent non-selective cannabinoid agonist, CP55,940.

This group confirmed that the cannabinoid ligand itself was incapable of inducing any proliferation of these B-cell subsets in the absence of CD40 MoAbs suggesting that CB2 receptors may act as co-receptors in the CD40-transduction pathway. They went further, however, to examine the distribution and modulation of CB2 receptors in the process of B-cell differentiation using RT-PCR to measure mRNA and flow cytometry. They found there was a dramatic downregulation of CB2 expression as B-cells left the virgin B-cell stage to become centroblasts but at the end of differentiation when memory B cells appeared, the CB2 receptor expression was restored.

Although cannabinoids alone are unable to induce proliferation of B-cell subsets, CB2 mRNA expression was upregulated in cultured splenocytes by anti-CD40 antibodies but was attenuated with the co-stimulation of IL-4. Later studies by the same group showed that there was a ten-fold difference in CB2 receptor expression between stimulated and unstimulated cultured B-cells treated with a nonspecific cannabinoid ligand, CP55940. This group showed that this nonselective cannabinoid increased IgE expression by 40-60% since it had previously been established that co-stimulation with IL-4 and anti-CD40 played a crucial role in the differentiation and class-switching to IgG1 and IgE in B cells. By using a selective CB2 antagonist, they concluded that CB2 activation is responsible for the class switching of IgM to IgE. However, the environment which influences isotype switching is complex and dependent on more than the presence of IL-4. This same group later found that a CB2 agonist was able to suppress an IgE response in mice. Other factors, as previously mentioned, such as the combination of IL-10 and TFG-β leads to the expression of IgA.

Based on the foregoing observations, it can be seen that activation of the endocannabinoid system with ligands selective for the receptors in the immune system (CB2) represents an opportunity for the development of an adjuvant for the administration of vaccines particularly for those epidermal, aerodigestive and sexually transmitted pathogens which are encountered through a mucosal route and direct contact with the skin. Accordingly, in one aspect disclosed herein are methods of enhancing an immune response (including, but not limited to a mucosal immune response to an ongoing infection) in a subject to an antigen (such as, for example a microbial antigen including, but not limited, to peptide, polypeptide, and/or protein antigens as well as live attenuated viruses, heat killed microbials, and genetically engineered microbes and microbial antigens used as vaccines or therapeutics) or ongoing infection comprising administering a cannabinoid receptor agonist (such as, for example L759,633; L759,656; JWH-056; JWH-133;JWH-229; JWH-352; JWH-359; THC; and/or CBD to the subject. For example, the immune responses that are enhanced can comprise increased and/or more rapid production of Immunoglobulin (Ig) A (IgA). In some aspects, the enhanced immune response further comprises an increase in production of B cell activating factor (BAFF/BLyS), a proliferation-inducing ligand (APRIL), Thymic stromal lymphopoietin (TSLP), IL-33, IL-10, IL-4, IL-6, and TGF-β.

It is understood and herein contemplated that by enhancing an immune response to an antigen (such as, for example a microbial antigen including, but not limited, to peptide, polypeptide, and/or protein antigens as well as live attenuated viruses, heat killed microbials, and genetically engineered microbes and microbial antigens used as vaccines or therapeutics) the efficacy of a vaccine can be increased. Thus, in one aspect, disclosed herein are methods of increasing the efficacy of a vaccine in a subject comprising administering a cannabinoid receptor agonist (such as, for example L759,633; L759,656; JWH-056; JWH-133;JWH-229; JWH-352; JWH-359; THC; and/or CBD) to the subject.

As noted throughout this disclosure, the disclosed methods and cannabinoid receptor agonists are particularly adept at enhancing and/or inducing mucosal immune responses (such as, for example, IgA production, and secretion of B cell activating factor (BAFF/BLyS), proliferation-inducing ligand (APRIL), Thymic stromal lymphopoietin (TSLP), IL-33, IL-10, IL-4, IL-6, and TGF-P.. Accordingly, in one aspect, disclosed herein are methods of inducing in a subject a mucosal immune response to an antigen said method comprising administering to the subject a cannabinoid receptor agonist (such as, for example L759,633; L759,656; JWH-056; JWH-133;JWH-229; JWH-352; JWH-359; THC; and/or CBD) and the antigen.

The disclosed cannabinoid receptor agonists are excellent adjuvants and thus can be used as part of a therapeutic or prophylactic vaccination regimen against a microbial infection. In one aspect, disclosed herein are methods of treating, inhibiting, reducing, decreasing, ameliorating, attenuating, and/or preventing a microbial infection in a subject comprising administering to a cannabinoid receptor agonist (such as, for example L759,633; L759,656; JWH-056; JWH-133;JWH-229; JWH-352; JWH-359; THC; and/or CBD) and an antigen; wherein the antigen comprises a microbial antigen.

The disclosed methods of enhancing an immune response; method of increasing the efficacy of a vaccine; methods of increasing the immunogenicity of an antigen; methods of inducing in a subject a mucosal immune response to an antigen; and/or methods of treating, inhibiting, reducing, decreasing, ameliorating, attenuating, and/or preventing a microbial infection in a subject comprise the use of cannabinoid receptor agonists, and more specifically cannabinoid receptor 2 agonists. Examples, of cannabinoid receptor 2 agonists comprise, but are not limited to the following:

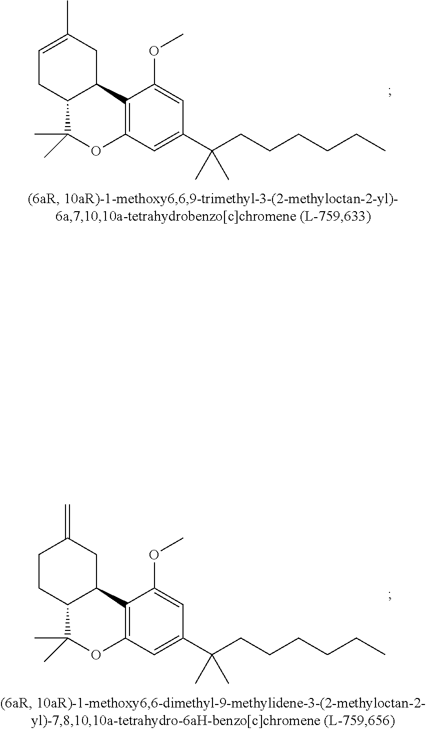

and Dibenzopyrans including, but no limited to the following; JWH-056

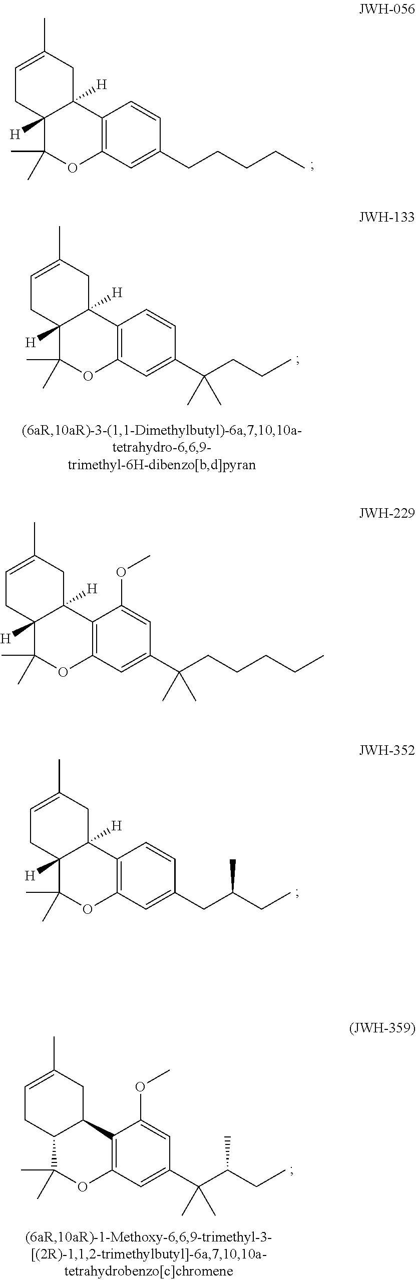

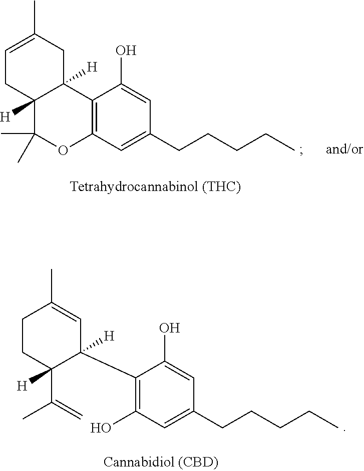 [SMILES: CCCCCc1cc(O)c2c(c1)OC(C)(C)[C@@H]1CC=C(C)C[C@@H]21 ; C=C(C)[C@@H]1CCC(C)=C[C@H]1c1c(O)cc(CCCCC)cc1O]

The disclosed methods of enhancing an immune response; method of increasing the efficacy of a vaccine; methods of increasing the immunogenicity of an antigen; methods of inducing in a subject a mucosal immune response to an antigen; and/or methods of treating, inhibiting, reducing, decreasing, ameliorating, attenuating, and/or preventing a microbial infection in a subject comprise the use of microbial antigens. As used herein microbial antigens can include, but are note limited to microbial peptides, polypeptides, and/or protein antigens, as well as, live attenuated viruses, heat killed microbials, and genetically engineered microbes and microbial antigens used as vaccines or therapeutics.

In one aspect, it is understood that the microbial antigen can be bacterial. Accordingly, disclosed herein are methods of enhancing an immune response; methods of increasing the efficacy of a vaccine; methods of increasing the immunogenicity of an antigen; methods of inducing in a subject a mucosal immune response to an antigen; and/or methods of treating, inhibiting, reducing, decreasing, ameliorating, attenuating, and/or preventing a microbial infection in a subject, wherein the antigen comprises a bacterial antigen from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Actinobacillus pleuropneumoniae, Listeria monocytogenes, Listeria ivanovii, Brucella abortus, other Brucella species, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Helicobactor pylori, Pseudomonas aeruginosa, other Pseudomonas species, Haemophilus influenzae (including, but not limited to Haemophilus influenzae type b), Haemophilus ducreyi, other Hemophilus species, Clostridium tetani, Clostridium difficile, Clostridium perfringens,other Clostridium species, Yersinia pestis, Yersinia enterolitica, and other Yersinia species.

In another aspect, it is understood that the microbial antigen can be viral. Accordingly, disclosed herein are methods of enhancing an immune response; methods of increasing the efficacy of a vaccine; methods of increasing the immunogenicity of an antigen; methods of inducing in a subject a mucosal immune response to an antigen; and/or methods of treating, inhibiting, reducing, decreasing, ameliorating, attenuating, and/or preventing a microbial infection in a subject, wherein the antigen comprises a viral antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A, Influenza virus B, Measles virus, Polyomavirus, Human Papilomavirus, Respiratory syncytial virus, Adenovirus, Coxsackie virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2

Also disclosed herein are methods of enhancing an immune response; method of increasing the efficacy of a vaccine; methods of increasing the immunogenicity of an antigen; methods of inducing in a subject a mucosal immune response to an antigen; and/or methods of treating, inhibiting, reducing, decreasing, ameliorating, attenuating, and/or preventing a microbial infection in a subject, wherein the antigen comprises a fungal antigen from a fungi selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

In one aspect, disclosed herein are methods of enhancing an immune response; method of increasing the efficacy of a vaccine; methods of increasing the immunogenicity of an antigen; methods of inducing in a subject a mucosal immune response to an antigen; and/or methods of treating, inhibiting, reducing, decreasing, ameliorating, attenuating, and/or preventing a microbial infection in a subject, wherein the antigen comprises an antigen from a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica

In some aspects the microbial antigen can be a commercially available vaccine. Accordingly, disclosed herein are methods of enhancing an immune response; method of increasing the efficacy of a vaccine; methods of increasing the immunogenicity of an antigen; methods of inducing in a subject a mucosal immune response to an antigen; and/or methods of treating, inhibiting, reducing, decreasing, ameliorating, attenuating, and/or preventing a microbial infection in a subject, wherein the antigen comprises a vaccine or an antigen from a portion of a vaccine selected from the group of vaccines to microbes including, but not limited to Vibrio cholerae (including, but not limited to DUKORAL1®, SHANCHOL®, EUVICHOL®, VAXCHORA®), Influenza type A and B virus (including, but not limited to FluMist™) Poliovirus (including, but not limited to Biopolio™ B1/3, and other oral polio vaccines—OPVs), Rotavirus (including, but not limited to ROTARIX® and ROTATEQ®), Salmonella typhimurium (including, but not limited to TYPHI VIVOTIF®), Adenovirus, Rabies virus (including, but note limited to RABORAL-V-RG), Bovine parainfluenza 3 (including, but not limited to Rispoval), bovine respiratory syncytial virus, Bordetella bronchiseptica (including, but not limited to NOBIVAC®), Canine parainfluenza virus, Newcastle disease virus (including, but not limited to Avinew NeO™), Diphtheria, Hepatitis B, Haemophilus influenzae type b, Human papillomavirus, Seasonal influenza, Measles, Mumps, Pertussis, Rubella, Pneumococcal disease, Poliomyelitis (Polio), Rotavirus, Tetanus, Tuberculosis (TB), Varicella, Cholera, Hepatitis A, Hepatitis E, Japanese encephalitis, Meningococcal disease, Polio (adult booster dose), Rabies, Tick-borne encephalitis, Typhoid fever, and/or Yellow fever.

As the timing of a microbial exposure can often not be predicted, it should be understood the disclosed methods of enhancing an immune response; method of increasing the efficacy of a vaccine; methods of increasing the immunogenicity of an antigen; methods of inducing in a subject a mucosal immune response to an antigen; and/or methods of treating, inhibiting, reducing, decreasing, ameliorating, attenuating, and/or preventing a microbial infection in a subject described herein can be used prior to (i.e., a prophylactic vaccine immunization) or following the onset of the disease or disorder (either before or after onset of symptoms), to treat, prevent, inhibit, and/or reduce the disease or disorder or symptoms thereof In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to the microbial infection; concurrently with the infection; 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18,24,30,36,48,60 hours; 3,4, 5,6,7, 8,9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months after the infection, but prior to onset of any symptoms of the infection; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18,24,30,36,48,60 hours; 3,4, 5,6,7, 8,9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the microbial infection or after onset of any symptoms of the infection.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

In one aspect, it is understood and herein contemplated that the microbial antigen and the cannabinoid receptor agonist (such as a cannabinoid receptor 2 agonist) can be administered as separate administrations that occur sequentially, separate administrations that occur concurrently, separate administrations that occur simultaneously, or as single administrative dose composition. Thus, in one aspect, the cannabinoid receptor agonist (such as a cannabinoid receptor 2 agonist) composition can be administered 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120 min, 3, 4, 5,6,7, 8,9, 10, 11, 12, 13, 14, 15, 16, 17, 18, 19, 20, 21, 22, 23, 24, 30, 36, 42, 48, 54, 60, 66, 72 hours, 4, 5, 6, 7, 8, 9, 10, 11, 12, 13, 14, 15, 16, 17, 18, 19, 20, 21, 22, 23, 24, 25, 26, 27, 28, 29, 30, 31, 32, 35, 42, 45, 49, 56, 58, 59, 60, 61, 62, 63, 90 days, 3, 4, 5, 6, 7, 8, 9, 10, 11, 12, 18, 24 months prior to administration of the microbial antigen or administered 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120 min, 3, 4, 5, 6, 7, 8, 9, 10, 11, 12, 13, 14, 15, 16, 17, 18, 19, 20, 21, 22, 23, 24, 30, 36, 42, 48, 54,60,66,72 hours, 4, 5,6,7, 8,9, 10, 11, 12, 13, 14, 15, 16, 17, 18, 19, 20, 21, 22, 23, 24, 25, 26, 27, 28, 29, 30, 31, 32, 35, 42, 45, 49, 56, 58, 59, 60, 61, 62, 63, 90 days, 3, 4, 5, 6, 7, 8, 9, 10, 11, 12, 18, 24 months following administration of the microbial antigen. As noted above, the cannabinoid and microbial antigen preparation can be administered concurrently, simultaneously, or as a combined single composition.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the anti-microbial vaccine or therapeutic, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, topically, buccal, rectal, vaginal, vaginal, nasal or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the anti-microbial vaccine or therapeutic. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular anti-microbial vaccine or therapeutic used, its mode of administration and the like. Thus, it is not possible to specify an exact amount for every composition. However, an appropriate amount can be determined by one of ordinary skill in the art using only routine experimentation given the teachings herein.

The materials may be in solution, suspension (for example, incorporated into microparticles,nanoparticles, liposomes, Archaeosomes, Bilosomes, ISCOM®, ISCOMATRIX®, Bacterial outer membrane vesicles (OMV), Virus-like particles (VLP), Gene gun (DNA vaccination), Emulsions Water-in-oil/oil-in-water, Synthetic polymer nanoparticles (e.g. PLA/PLGA), Natural polymer nanoparticles (e.g. chitosan), Hydrogel (e.g. cCHP nanogel), Lactic acid bacteria (LAB), Chemically processed pollen grains (PGs), Terrestrial plants and algae, or virosomes These may be targeted to a particular cell type via receptors, or receptor ligands.

### a) Pharmaceutically Acceptable Carriers

The compositions, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the disclosed CB2 agonist compositions including anti-microbial vaccines or therapeutics, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice.

The pharmaceutical composition may be administered in a number of ways depending on whether local or systemic treatment is desired, and on the area to be treated. Administration may be topically (including ophthalmically, vaginally, rectally, intranasally), orally, by inhalation.

Preparations for administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

### C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular combination of microbial antigen and cannabinoid receptor agonist is disclosed and discussed and a number of modifications that can be made to a number of molecules including the microbial antigen and cannabinoid receptor agonist are discussed, specifically contemplated is each and every combination and permutation of microbial antigen and cannabinoid receptor agonist and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect, disclosed are anti-microbial vaccine or therapeutic comprising a microbial antigen and a cannabinoid receptor agonist (such as, for example, a cannabinoid receptor 2 agonist including, but not limited to L759,633; L759,656; JWH-056; JWH-133;JWH-229; JWH-352; JWH-359; THC; and/or CBD.

Also disclosed herein are anti-microbial vaccines or therapeutics, wherein the microbial antigen comprises a bacterial antigen from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Actinobacillus pleuropneumoniae, Listeria monocytogenes, Listeria ivanovii, Brucella abortus, other Brucella species, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Helicobactor pylori, Pseudomonas aeruginosa, other Pseudomonas species, Haemophilus influenzae (including, but not limited to Haemophilus influenzae type b), Haemophilus ducreyi, other Hemophilus species, Clostridium tetani, Clostridium difficile, Clostridium perfringens, other Clostridium species, Yersinia pestis, Yersinia enterolitica, and other Yersinia species.

In one aspect, disclosed herein are anti-microbial vaccines or therapeutics, wherein the microbial antigen comprises a viral antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A, Influenza virus B, Measles virus, Polyomavirus, Human Papilomavirus, Respiratory syncytial virus, Adenovirus, Coxsackie virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed herein are anti-microbial vaccines or therapeutics, wherein the microbial antigen comprises a fungal antigen from a fungi selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

Also disclosed herein are anti-microbial vaccines or therapeutics, wherein the microbial antigen comprises an antigen from a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

### D. References

Agudelo, M., Newton, C., Widen, R., Sherwood, T., Nong, L., Friedman, H. & Klein, T. W. 2008, ‘Cannabinoid receptor 2 (CB2) mediates immunoglobulin class switching from IgM to IgE in cultures of murine-purified B lymphocytes’, J Neuroimmune Pharmacol, vol. 3, no. 1, pp. 35-42.
Basu, Sreemanti et al. “Cannabinoid Receptor 2 (CB2) Plays a Role in the Generation of Germinal Center and Memory B Cells, but Not in the Production of Antigen-Specific IgG and IgM, in Response to T-dependent Antigens.” PloS one vol. 8,6 e67587. 27 Jun. 2013, doi:10.1371/journal.pone.0067587
Börner C, Smida M, Höllt V, Schraven B, Kraus J. Cannabinoid receptor type 1- and 2-mediated increase in cyclic AMP inhibits T cell receptor-triggered signaling. J Biol Chem. 2009 Dec. 18;284(51):35450-60. doi: 10.1074/jbc.M109.006338. PMID: 19858202; PMCID: PMC2790974.
Carayon, P., Marchand, J., Dussossoy, D., Derocq, J. M., Jbilo, O., Bord, A., Bouaboula, M., Galiegue, S., Mondiere, P., Penarier, G., Fur, G. L., Defrance, T. & Casellas, P. 1998, ‘Modulation and functional involvement of CB2 peripheral cannabinoid receptors during B-cell differentiation’, Blood, vol. 92, no. 10, pp. 3605-3615.
Chen Z M, O'Shaughnessy M J, Gramaglia I, Panoskaltsis-Mortari A, Murphy W J, Narula S, Roncarolo M G, Blazar B R. IL-10 and TGF-beta induce alloreactive CD4+CD25-T cells to acquire regulatory cell function. Blood. 2003 Jun. 15;101(12):5076-83.
Coopman K, Smith L D, Wright K L, Ward S G. Temporal variation in CB2R levels following T lymphocyte activation: evidence that cannabinoids modulate CXCL12-induced chemotaxis. Int Immunopharmacol. 2007 Mar.; 7(3):360-71.
Correa F, Mestre L, Docagne F, Guaza C. Activation of cannabinoid CB2 receptor negatively regulates IL-12p40 production in murine macrophages: role of IL-10 and ERK1/2 kinase signaling. Br J Pharmacol. 2005 June; 145(4):441-8.
Cushman P, Khurana R Marijuana and T lymphocyte rosettes. Clin Pharmacol Ther. 1976 Mar.; 19(3):310-7.
Defrance, T. et al. Interleukin 10 and transforming growth factor beta cooperate to induce anti-CD40-activated naive human B cells to secrete immunoglobulin A. J Exp Med 175, 671-682 (1992).
Derocq J M, Ségui M, Marchand J, Le Fur G, Casellas P. 1995 Cannabinoids enhance human B-cell growth at low nanomolar concentrations. FEBS Lett. Aug. 7;369(2-3):177-82.
Feng R, Milcarek C A, Xie X Q. Antagonism of cannabinoid receptor 2 pathway suppresses IL-6-induced immunoglobulin IgM secretion. BMC Pharmacol Toxicol. 2014 Jun. 9; 15:30. doi: 10.1186/2050-6511-15-30. PMID: 24913620; PMCID: PMC4062519.
Galiegue, S., Mary, S., Marchand, J., Dussossoy, D., Carriere, D., Carayon, P., Bouaboula, M., Shire, D., Le Fur, G. & Casellas, P. 1995, ‘Expression of central and peripheral cannabinoid receptors in human immune tissues and leukocyte subpopulations’, Eur J Biochem, vol. 232, no. 1, pp. 54-61.
Gardner B, Zu L X, Sharma S, Liu Q, Makriyannis A, Tashkin D P, Dubinett S M. Autocrine and paracrine regulation of lymphocyte CB2 receptor expression by TGF-beta. Biochem Biophys Res Commun. 2002 Jan. 11;290(1):91-6.
Ghosh S, Preet A, Groopman J E, Ganju R K. Cannabinoid receptor CB2 modulates the CXCL12/CXCR4-mediated chemotaxis of T lymphocytes. Mol Immunol. 2006 July; 43(14):2169-79.
Ibsen M S, Connor M, Glass M. Cannabinoid C B₁ and CB₂ Receptor Signaling and Bias. Cannabis Cannabinoid Res. 2017 Mar. 1;2(1):48-60. doi: 10.1089/can.2016.0037. PMID: 28861504; PMCID: PMC5436336
Jan T R, Su S T, Wu H Y, Liao M H. Suppressive effects of cannabidiol on antigen-specific antibody production and functional activity of splenocytes in ovalbumin-sensitized BALB/c mice. Int Immunopharmacol. 2007 Jun.; 7(6):773-80. doi: 10.1016/j.intimp.2007.01.015. Epub 2007 Feb. 15. PMID: 17466911.
Jorda M A, Verbakel S E, Valk P J, Vankan-Berkhoudt Y V, Maccarrone M, Finazzi-Agro A, Lowenberg B, Delwel R. Hematopoietic cells expressing the peripheral cannabinoid receptor migrate in response to the endocannabinoid 2-arachidonoylglycerol. Blood. 2002 Apr. 15;99(8):2786-93.
Kaneko, Y., Hirose, S., Abe, M., Yagita, H., Okumura, K. & Shirai, T. 1996, ‘CD40-mediated stimulation of B1 and B2 cells: implication in autoantibody production in murine lupus’, Eur J Immunol, vol. 26, no. 12, pp. 3061-3065.
Kishimoto S, Gokoh M, Oka S, Muramatsu M, Kajiwara T, Waku K, Sugiura T. 2-arachidonoylglycerol induces the migration of HL-60 cells differentiated into macrophage-like cells and human peripheral blood monocytes through the cannabinoid CB2 receptor-dependent mechanism. J Biol Chem. 2003 Jul. 4;278(27):24469-75.
Kishimoto S, Muramatsu M, Gokoh M, Oka S, Waku K, Sugiura T. Endogenous cannabinoid receptor ligand induces the migration of human natural killer cells. J Biochem (Tokyo). 2005 Feb.; 137(2):217-23.
Klein T W Cannabinoid-based drugs as anti-inflammatory therapeutics.Nat Rev Immunol. 2005 May; 5(5):400-11. Review.
Klein T W, Newton C, Larsen K, Chou J, Perkins I, Lu L, Nong L, Friedman H. Cannabinoid receptors and T helper cells. J Neuroimmunol. 2004 February; 147(1-2):91-4.
Lee, S. F., Newton, C., Widen, R., Friedman, H. & Klein, T. W. 2001, ‘Differential expression of cannabinoid CB(2) receptor mRNA in mouse immune cell subpopulations and following B cell stimulation’, Eur J Pharmacol, vol. 423, no. 2-3, pp. 235-241.
Lim V Y, Zehentmeier S, Fistonich C, Pereira J P. A Chemoattractant-Guided Walk Through Lymphopoiesis: From Hematopoietic Stem Cells to Mature B Lymphocytes. Adv Immunol. 2017; 134:47-88. doi: 10.1016/bs.ai.2017.02.001. Epub 2017 Mar. 18. PMID: 28413023; PMCID: PMC5784265.
Lu H, Crawford R B, North C M, Kaplan B L, Kaminski N E. Establishment of an immunoglobulin m antibody-forming cell response model for characterizing immunotoxicity in primary human B cells. Toxicol Sci. 2009 December; 112(2):363-73. doi: 10.1093/toxsci/kfp224. Epub 2009 Sep. 18. PMID: 19767444; PMCID: PMC2777081.
Maestroni G J. The endogenous cannabinoid 2-arachidonoyl glycerol as in vivo chemoattractant for dendritic cells and adjuvant for Th1 response to a soluble protein. FASEB J. 2004 Dec.; 18(15):1914-6.
Mestecky J, Russell M W, Elson C O. Perspectives on mnucosal vaccines: is mucosal tolerance a barrier? J Immunol (2007) 179(9):5633-8. doi: 10.4049/jiinmunol.179.9.5633
Miquel-Clopés A, Bentley E G, Stewart J P, Carding S R. Mucosal vaccines and technology. Clin Exp ninmunol. 2019 May:196(2):205-214, doi: 10.111/cei.13285, Epub 2019 Apr. 8. PMID: 30963541; PMCID: PMC6468177.
Muppidi, Jagan R et al. “Cannabinoid receptor 2 positions and retains marginal zone B cells within the splenic marginal zone.” The Journal of experimental medicine vol. 208,10 (2011): 1941-8. doi:10.1084/jem.20111083
Newton C A, Chou P J, Perkins I, Klein T W. 2009 CB(1) and CB(2) cannabinoid receptors mediate different aspects of delta-9-tetrahydrocannabinol (THC)-induced T helper cell shift following immune activation by Legionella pneumophila infection. J Neuroimmune Pharmacol. Mar.; 4(1):92-102.
Newton, C. A., Chou, P. J., Perkins, I. and Klein, T. W. (2009) CB(1) and CB(2) cannabinoid receptors mediate different aspects of delta-9-tetrahydrocannabinol (THC)-induced T helper cell shift following immune activation by Legionella pneumophila infection. J Neuroimmune Pharmacol 4: 92-102.
Nikolova E B, Tomana M, Russell M W. All forms of human IgA antibodies bound to antigen interfere with complement (C3) fixation induced by IgG or by antigen alone. Scand J Immunol (1994) 39(3):275-80. doi: 10.111/j. 1365-3083.1994.tb03371x
Pacifici R, Zuccaro P, Pichini S, Roset P N, Poudevida S, Farre M, Segura J, De la Torre R. Modulation of the immune system in cannabis users. JAMA. 2003 Apr. 16;289(15):1929-31.
Pereira J P, An J, Xu Y, Huang Y, Cyster J G. Cannabinoid receptor 2 mediates the retention of immature B3 cells in bone marrow sinusoids. Nat Immunol. 2009 April; 10(4):403-11. doi: 10.1038/ni.1710. Epub 2009 Mar. 1. PMID: 19252491; PMCID: PMC2768754.
Plotkin S A. Correlates of protection induced by vaccination. Clin Vaccine immunol 2010 Jul.; 17(7):1055-65. doi: 10.1128/CV1.00131-10. Epub 2010 May 12. PMID: 20463105; PMCID: PMC2897268.
Rayman N. Lam K H, Laman J D, Simons P J, Löwenberg B, Sonneveld P, Delwel R. Distinct expression profiles of the peripheral cannabinoid receptor in lymphoid tissues depending on receptor activation status. J Immunol. 2004 Feb. 15;172(4):2111-7. doi: 10.4049/jimmunol.172.4.2111 PMID: 14764676.
Russell M W, Sibley D A, Nikolova F B, Tomana M, Mestecky J. IgA antibody as a non-inflammatory regulator of immunity. Biochem Soc Trans (1997) 25(2):466-70, doi: 10.1042/bst0250466
Saroz Y, Kho D T, Glass M, Graham E S, Grimsey N L. Cannabinoid Receptor 2 (CB₂) Signals via G-alpha-s and Induces I L-6 and IL-10 Cytokine Secretion in Human Primary Leukocytes. ACS Pharmacol Transl Sci. 2019 Oct. 1;2(6):414-428. doi: 10.1021/acsptsci.9b00049. PMID: 32259074; PMCID: PMC7088898.
Soethoudt M, Grether U, Fingerle J, Grim T W, Fezza F, de Petrocellis L, Ullmer C, Rothenhäusler B, Perret C, van Gils N, Finlay D, MacDonald C., Chicca A. Gens M D, Stuart J, de Vries H, Mastrangelo N, Xia L, Alachouzos G, Baggelaar M P, Martella A, Mock E D, Deng H, Heitman L H, Connor M, Di Marzo V, Gertsch J, Lichtman A H, Maccarrone M, Pacher P, Glass M, van der Stelt M. Cannabinoid CB2 receptor ligand profiling reveals biased signalling and off-target activity. Nat Commun. 2017 Jan. 3; 8:13958. doi: 10.1038/ncomms13958. PMID: 28045021; PMCID: PMC5216056.
Tang J L, Lancz G, Specter S. 1993 Delta-9-tetrahydrocannabinol-(THC)-mediated inhibition of macrophage macromolecular metabolism is antagonized by human serum proteins and by cell surface proteins. Int J Immunopharmacol. Aug.; 15(6):665-72.
Travis C R. As Plain as the Nose on Your Face: The Case for A Nasal (Mucosal) Route of Vaccine Administration for Covid-19 Disease Prevention. Front Immunol. 2020 Sep. 30; 11:591897. doi: 10.3389/fimmu.2020.591897. PMID: 33117404; PMCID: PMC7561361.
Woof, J. M. & Mestecky, J. Mucosal immunoglobulins. Immunol Rev 206, 64-82 (2005).
Yeh C Y, Yeh T H, Jung C J, Chen P L, Lien H T, Chia J S. Activated human nasal epithelial cells modulate specific antibody response against bacterial or viral antigens. PLoS One. 2013;8(2):e55472. doi: 10.1371/journal.pone.0055472. Epub 2013 Feb. 6. PMID: 23405156; PMCID: PMC3566203.
Yuan M, Kiertscher S M, Cheng Q, Zoumalan R, Tashkin D P, Roth M D Delta 9-Tetrahydrocannabinol regulates Th1/Th2 cytokine balance in activated human T cells. J Neuroimmunol. 2002 December; 133(1-2):124-31.
Zheng S G, Wang J H, Gray J D, Soucier H, Horwitz D A. Natural and induced CD4+CD25+cells educate CD4+CD25-cells to develop suppressive activity: the role of IL-2, TGF-beta, and IL—. J Immunol. 2004 May 1;172(9):5213-21.
Ziring D, Wei B, Velazquez P, Schrage M, Buckley N E, Braun J. Formation of B and T cell subsets require the cannabinoid receptor CB2. Immunogenetics. 2006 September; 58(9):714-25.

What is claimed is:

1. A method of enhancing or inducing an immune response in a subject to an antigen comprising administering a cannabinoid receptor agonist to the subject.

2. The method of enhancing an immune response of claim 1, wherein the immune responses that is enhanced is a mucosal immune response.

3. The method of enhancing an immune response of claim 2, wherein the enhanced immune response further comprises an increase in production of B cell activating factor (BAFF/BLyS), a proliferation-inducing ligand (APRIL), Thymic stromal lymphopoietin (TSLP), IL-33, IL-10, IL-4, IL-6, and TGF-β.

4. The method of enhancing an immune response of claim 1, wherein the immune responses that is enhanced comprises increased and/or more rapid production of Immunoglobulin (Ig) A (IgA).

5. The method of claim 1, wherein the cannabinoid receptor agonist and the antigen are administered in the same formulation.

6. The method of claim 1, wherein the cannabinoid receptor agonist and the antigen are administered concurrently.

7. The method of claim 1, wherein the cannabinoid receptor agonist is administered before administration of the antigen.

8. The method of claim 1, wherein the cannabinoid receptor agonist is administered after administration of the antigen.

9. The method of claim 1, wherein the antigen comprises a bacterial antigen from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Actinobacillus pleuropneumoniae, Listeria monocytogenes, Listeria ivanovii, Brucella abortus, other Brucella species, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Helicobactor pylori, Pseudomonas aeruginosa, other Pseudomonas species, Haemophilus influenzae (including, but not limited to Haemophilus influenzae type b), Haemophilus ducreyi, other Hemophilus species, Clostridium tetani, Clostridium difficile, Clostridium perfringens,other Clostridium species, Yersinia pestis, Yersinia enterolitica, and other Yersinia species.

10. The method of claim 1, wherein the antigen comprises a viral antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A, Influenza virus B, Measles virus, Polyomavirus, Human Papilomavirus, Respiratory syncytial virus, Adenovirus, Coxsackie virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

11. The method of claim 1, wherein the antigen comprises a fungal antigen from a fungi selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

12. The method of claim 1, wherein the antigen comprises an antigen from a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

13. The method of any of claim 1, wherein the cannabinoid comprises a cannabinoid receptor 2 agonist.

14. The method of claim 12, wherein the cannabinoid receptor 2 agonist comprises L759,633; L759,656; JWH-056; JWH-133; JWH-229; JWH-352; JWH-359; THC; and/or CBD.

15. An anti-microbial vaccine or therapeutic comprising a microbial antigen and a cannabinoid receptor 2 agonist.

16. The anti-microbial vaccine or therapeutic of claim 15, wherein the microbial antigen comprises a bacterial antigen from a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Actinobacillus pleuropneumoniae, Listeria monocytogenes, Listeria ivanovii, Brucella abortus, other Brucella species, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Helicobactor pylori, Pseudomonas aeruginosa, other Pseudomonas species, Haemophilus influenzae, Haemophilus ducreyi, other Hemophilus species, Clostridium tetani, Clostridium difficile, Clostridium perfringens,other Clostridium species, Yersinia pestis, Yersinia enterolitica, and other Yersinia species.

17. The anti-microbial vaccine or therapeutic of claim 15, wherein the microbial antigen comprises a viral antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV), Influenza virus A, Influenza virus B, Measles virus, Polyomavirus, Human Papilomavirus, Respiratory syncytial virus, Adenovirus, Coxsackie virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

18. The anti-microbial vaccine or therapeutic of claim 15, wherein the microbial antigen comprises a fungal antigen from a fungi selected from the group consisting of Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

19. The anti-microbial vaccine or therapeutic of claim 15, wherein the microbial antigen comprises an antigen from a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

20. The anti-microbial vaccine or therapeutic of claim 22, wherein the cannabinoid receptor 2 agonist comprises L759,633; L759,656; JWH-056; JWH-133;JWH-229; JWH-352; JWH-359; THC; and/or CBD.

21. A method of treating a microbial infection in a subject comprising administering to the subject anti-microbial vaccine or therapeutic of claim 15.

22. A method of increasing the immunogenicity of an antigen or increasing the efficacy of a vaccine comprising administering a cannabinoid receptor agonist with the antigen.
